(19) United States
(12) Patent Application Publication
(10) Pub. No.: US 2008/0187511 A1
(43) Pub. Date: Aug. 7, 2008

(54) OINTMENT FOR CANCER TREATMENT

(75) Inventors: Michael R. Shurin, Wexford, PA (US); Valerian E. Kagan, Pittsburgh, PA (US); Galina V. Shurin, Wexford, PA (US); Yulia Tyurina, Pittsburgh, PA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignees: University of Pittsburgh-Of the Commonwealth; System of Higher Education, Pittsburgh, PA (US)

(21) Appl. No.: 11/924,354

(22) Filed: Oct. 25, 2007

Related U.S. Application Data
(60) Provisional application No. 60/854,273, filed on Oct. 25, 2006.

(51) Int. Cl. A61K 38/19; A61K 31/685; A61K 31/683; A61P 35/00

(52) U.S. Cl. 424/85.1; 514/114; 514/134

(57) ABSTRACT

The invention provides an oil-based pharmaceutical composition comprising as an active ingredient phostphatidylserine or a derivative thereof and a pharmaceutically acceptable oil-based carrier, which is formulated for topical or intratumoral administration. Preferably, the composition is an ointment or a cream. The composition can contain additional active agents as well, such as aminophospholipid translocase inhibitors, transnitrosylating agents, chemokines, cytokines, Toll-like receptor ligands, imidazoquinolines, dendritic cell differentiation factors, or combinations thereof. In another aspect, the invention provides a method of treating cancer within a patient in need of treatment by administering the inventive composition to the patient.

This application claims priority to U.S. Provisional Patent Application 60/854,273, filed Oct. 25, 2006, the entire contents of which are incorporated herein in their entirety.

### BACKGROUND OF THE INVENTION

Nationally, there are more new cases of skin cancer each year than the combined incidence of cancers of the breast, prostate, lung, and colon with more than 1.5 million skin cancers diagnosed yearly in the United States. One in 5 Americans and one in 3 Caucasians will develop skin cancer in the course of a lifetime. Skin cancer is the most prevalent cancer in men over age 50, ahead of prostate, lung and colon cancer. Thus novel, especially non-invasive therapeutic approaches are highly justified.

Phosphatidylserine (PS) plays a key role in recognition of dying tumor cells by dendritic cells (DC) and thus is involved in initiation and promotion of natural antitumor immune response. Chen et al. reported for the first time that PS is specifically recognized via PSR expressed on the surface of DC and triggers phagocytosis [Chen et al., J Immunol 2004; 173:2985-2994]. Following their encounter with apoptotic cells, DC can be rendered immunologically inert, immunosuppressive or immunostimulatory [Ip et al., J Immunol 2004; 173:189-196], [Morelli, Blood 2003; 101:611-620.][Voll et al., Nature 1997; 390:350-351]. Also, it has been reported [Sauter et al., J Exp Med 2000; 191:423-434; Wong et al., Immunology 2005; 116:13-20] that necrotic cells can modulate immune responses of DC. These contradictory findings have been difficult to resolve due to the complexity of receptors on DC. For instance, while αvβ5 mediates efficient phagocytosis, it does not interfere with the DC capacity to undergo maturation or stimulate T cells. In contrast, engagement of CR3 (or CR4) inhibits the ability of DC to undergo maturation, produce pro-inflammatory cytokines or chemolcines and activate T cells [Skobeme, Blood. 2006 Aug. 1; 108(3):947-55. Epub 2006 Apr. 13].

The results of PS-mediated signaling in DC are also contradictory. Girolomoni et al. have reported that PS is capable of up-regulating the induction of contact hypersensitivity in mice by stimulating the APC function of epidermal DC [Girolomoni, J Immunol 1993; 150:4236-4243]. This effect can be explained by the fact that PS and other phospholipids enhance the binding of peptides to MHC class II molecules [Roof, Proc Natl Acad Sci USA 1990; 87:1735-1739]. Different results were published by Chen et al. who suggested that PS inhibits the ability of DC to undergo maturation, secrete IL-12, activate T cells, and stimulate IFN-γ-producing CD4+ T cells [Chen et al., J Immunol 2004; 173:2985-2994]. However, all these data were obtained exclusively by studying the effects of PS-containing liposomes on DC.

With the recent emergence of lipid-based delivery vehicles of different drugs to tumor cells, PS presents a potentially promising and feasible tool for cancer therapy. For example, Francois et al. (Published International Patent Application WO 2005/019429) have proposed “tagging” cells with PS either through conjugating PS (or derivatives thereof) with an antibody or cell-surface ligand (see, e.g., page 22, paragraph 84) for delivering the PS to the cell surface, or formulating PS (or derivatives thereof) into a fusogenic liposome, which likewise preferably comprises an antibody or cell-surface ligand for targeting the liposome to the cell surface (see page 29, paragraph 103). This approach seeks to promote phagocytosis. However, clinical use of targeting agents such as antibodies can be problematic in some patients and requires antibodies to predefined tumor markers, which are rare for certain types of cancer (notably melanomas). Accordingly, there remains a need for a readily applied formulation for treatment of cancers, particularly a noninvasive topical immunotherapeutic approach for the treatment of skin cancer.

### BRIEF SUMMARY OF THE INVENTION

The invention provides an oil-based pharmaceutical composition comprising as an active ingredient phostphatidylserine (PS) or a derivative thereof and an oil-based pharmaceutically acceptable carrier, which is formulated for topical administration. Preferably, the composition is an ointment or a cream. The composition can contain additional active agents as well, such as aminophospholipid translocase inhibitors, transnitrosylating agents, chemokines, cytokines, Toll-like receptor ligands, imidazoquinolines, dendritic cell differentiation factors, or combinations thereof. In another aspect, the invention provides a method of treating cancer within a patient in need of treatment by administering the inventive composition to the patient. These aspects and other inventive features will be apparent upon reviewing the accompanying figures and from the following detailed description.

### BRIEF DESCRIPTION OF THE SEVERAL VIEWS OF THE DRAWING(S)

FIG. 1. Phospholipid composition of mouse B16 melanoma cells. Tumor-bearing mice received topical skin application of the PS-ointment over the growing s.c. melanoma for 3 days (ointment) or no treatment (control). The tumors were removed, digested and total lipids were extracted and analyzed as described in the text. FIG. 1A depicts typical 2D-HPTLC of total lipids extracted from in vivo growing B 16 melanoma cells. FIG. 1B graphically presents quantitative analysis of phospholipids in treated and non-treated melanoma cells. Both PS and PC were markedly increased in tumor cells treated with the ointment containing PC:PS=1:1. (N=3). NL, neutral lipids; FFA, free fatty acids; CL, cardiolipin; PE, phosphatidylethanolamine; PC, phosphatidylcholine; PS, phosphatidylserine; PI, phosphatidylinositol; Sph, sphingomyelin; LPC, lysophosphatidylcholine.

FIG. 2 graphically presents data concerning topical application of the PS ointment caused significant inhibition of B 16 tumor growth in mice. Melanoma cells were inoculated s.c. and the topical phospholipids-ointment-based therapy was initiated 24 h later. The ointments were applied for 2 weeks and the tumor size as determined every other day with a caliper.

FIG. 3 graphically presents data concerning topical application of the PS-ointment in B16-bearing mice caused the gene-ration of B16-specific T cells in the spleen. T cells were isolated from the spleens obtained from B 16-bearing mice treated with phospholipid-containing ointments. Cells were stimulated with medium or irradiated B16 or RM1 cells and IL-2 and IFN-γ were assessed 24 h later.

FIG. 4 graphically presents the time-course of changes of intratumoral cytokines in B16-bearing mice treated with phospholipids-containing ointment.

FIG. 5 graphically presents data demonstrating that PS-ointment failed to inhibit tumor growth in SCID mice. Immunodeficient SCID mice were treated with the PS-ointment for 3 weeks starting 24 h after B 16 cell inoculation s.c. Control mice received no therapy (3 mice/group). The results from two independent experiments are shown.

FIG. 6 graphically presents data demonstrating that PS-ointment based precipitated an antitumor effect, i.e. inhibited growth of untreated tumor located on the other side of the body. Immunocompetent syngeneic C57BL6 mice received topical therapy with the PS-ointment on the left flank for 2 weeks starting 24 h after s.c. inoculation of B16 cells in both left and right flanks. Control mice received no therapy. The results from two independent experiments are shown.

FIG. 7 graphically presents data demonstrating that the PS-ointment displays no antitumor activity in IFN-γ knockout mice.

### DETAILED DESCRIPTION OF THE INVENTION

In one aspect, the invention provides an oil-based pharmaceutical composition comprising as an active ingredient PS, a derivative thereof, or combinations thereof. In this context, a derivative of PS is a chemically-modified form of PS that, when present on the surface of cells, can precipitate an immune response from DC. However, the term does not include conjugation with moieties, such as cell-surface ligands, antibodies, and the like, that serve as targeting agents.

Preferred derivatives of PS include derivatives that comprise two saturated fatty acids, saturated and monounsaturated (oleic, C18:1), or polyunsaturated fatty acids (linoleic C18:2, linolenic C18:3, arachidonic C20:4, docosahexaenoic C20:4). Other preferred derivatives of PS for use in the present invention include oxidized form of phostphatidylserine (such as arachidonoyl(OX)-oleoyl-phostphatidylserine) (available from sources such as Avanti Polar Lipids (Alabaster, Ala.); see also Tyurina et al., Biochem Biophys Res Commun. 2004, 324(3): 1059-64). Additional preferred oxidized PS species carry an oxidatively truncated sn-2 acyl group with a terminal γ-hydroxy(or oxo)-α,β-unsaturated carbonyl (see, e.g., Podrez, et al., J. Biol. Chem. 277:38517-38523 (2002) and Podrez et al., J. Biol. Chem. 277:38503-38516 (2002)). Additional oxidized PS derivatives of interest, which can be used in the inventive method and composition, include those described in Table 1 (Greenberg et al., J. Exp. Med., 203(12), 2613-2625 (2006)).

Lyso forms of PS and such derivatives also are acceptable for use in the inventive composition. Lyso forms can be prepared by hydrolyzing the fatty acid, such as with phospholipase A2 or by other methods known to those of skill in the art. Other derivatives of PS that can be suitably employed in the context of the present invention (except for forms that are conjugated to antibodies, ligands, and the like) are described in Francois et al. (Published International Patent Application WO 2005/019429), the disclosure of which is incorporated herein by reference.

TABLE I
Parent ([M − H]⁻) and characteristic daughter ions (MS2
and MS3) of candidate CD36 ligands from oxidized PAPS and PLPS

| | | | | |
|---|---|---|---|---|
| m/z | MS2 | MS3 | Structure of sn-2 side chain | Abbreviations |

PL-PS oxidation products

| | | | | |
|---|---|---|---|---|
| 650.3 | 563.3 | 409/391/307/255/171/153 | 9-oxononanoic acid | ON-PS |
| 666.3 | 579.3 | 409/391/323/255/187/153 | Azeleic acid | A-PS |
| 704.3 | 617.3 | 409/391/361/255/225/153 | 9,12-dioxododec-10-enoic acid | KODA-PS |
| 706.3 | 619.3 | 409/391/363/255/227/153 | 9-hydroxy-12-oxodedec-10-enoic acid | HODA-PS |
| 720.3 | 633.3 | 409/391/377/255/241/153 | 9-oxo-11-carboxyundee-6-enoic acid | KDdiA-PS |
| 722.3 | 635.3 | 409/391/379/255/243/153 | 9-hydroxy-11-carboxyundec-6-enoic acid | HDdiA-PS |
| 758.3 | 671.3 | | Linoleic acid | PL-PS |

PA-PS oxidation products

| | | | | |
|---|---|---|---|---|
| 594.3 | 507.3 | 409/391/255/251/153/115 | 5-oxovaleric acid | OV-PS |
| 610.3 | 523.3 | 409/391/267/255/153/131 | Glutaric acid | G-PS |
| 648.3 | 561.3 | 409/391/305/255/169/153 | 5,8-dioxooct-6-enoic acid | KOOA-PS |
| 650.3 | 563.3 | 409/391/307/255/153 | 5-hydroxy-8-oxooct-6-enoic acid | HOOA-PS |
| 664.3 | 577.3 | 409/391/321/255/184/153 | 5-oxo-7-carboxyhept-6-enoic acid | KOdiA-PS |
| 666.3 | 579.3 | 409/391/323/255/187/153 | 5-hydroxy-7-carboxyhept-6-enoic acid | HOdiA-PS |
| 782.3 | 695.3 | | Arachidonic acid | PA-PS |

Abbreviations: PAPS, 1-palmitoyl-2-arachidenoyl-sn-glycero-3-phosphoserine; PLPS, 1-palmitoyl-2-ineoyl-sn-glycero-3-phosphoserine; oxPS, oxidized PS.
According to Greenberg et al., J. Exp. Med., 203(12), 2613-2625 (2006)), PAPS and PLPS were individually oxidized and analyzed by HPLC with on-line electrospray ioniz(ation mass spectrometry on an iontrap instrument using MS mode. Parent and characteristic MS2 and MS3 daughter ions (for sn-2 oxfatty acid) are shown. Ions in the MS2 spectrum that are produced after neutral loss (m/z = 87) of aziridine-2- carboxylic acid from the precursor parent ions were optimized and used as the precursor for MS3 experiments. The MS3 ions of the corresponding sn-2 oxidized fatty acids previously identified as CD36 ligands within oxPC species (Podrez, et al., J. Biol. Chem. 277: 38517-38523 (2002) and Podrez et al., J. Biol. Chem. 277: 38503-38516 (2002)) were isolated and fragmented. The structure and observed ions of the sn-2 chain of oxPS are shown. These corresponded to the previously identified sn-2 side chains on oxPC that serve as CD36 ligands.

Any suitable amount of PS (and/or PS-derivative) can be included in the inventive composition. However, typically, the amount of PS (and/or PS-derivative) is adjusted to deliver PS (and/or PS-derivative) to the patient in a dosage between about 10 μg/cm²/day and about 75 μg/cm²/day when administered topically (e.g., the “cm²” refers to the area of the patient's skin). More preferably, the dosage of PS is between about 12 μg/cm²/day and about 60 μg/cm²/day, and even more preferably less than about 50 μg/cm²/day (such as about 25 μg/cm²/day or between about 30 μg/cm²/day and about 40 μg/cm²/day). To achieve such dosages, the PS or derivative thereof typically comprises between about 1 wt % and about 20 wt % of the composition, such as about 3 wt % (30 mg/g) or about 6 wt % (60 mg/g) or about 10 wt % (100 mg/g) or about 15 wt % (150 mg/g).

The oil-based carrier for inclusion in the inventive composition should include an agent able to enhance the penetration of the PS (and/or PS-derivative) through the skin, such as a fatty acid or oil. In this manner, the carrier is not water-soluble. For example, oleic acid, mono-unsaturated oleic acid (C18:1) or a polyunsaturated fatty acid (such as linoleic acid (C18:2), linolenic acid (C18:3) or arachidonic acid), or combinations thereof can be employed within the carrier. Of these, monounsaturated fatty acids, such as oleic acid are preferred, as they are stable due to is low susceptibility to peroxidation. However, where oxidized PS derivatives are employed, then inclusion of polyunsaturated fatty acids can advantageously be included in the carrier to promote PS oxidation. However, oils such as soybean oil are less preferred for use in the inventive composition, as such can decrease lymphocyte proliferation and provoke neutrophil and lymphocyte apopotosis and necrosis (Cury-Boaventura et al., J Parenter Enteral Nutr. 2006 March-April; 30(2): 115-23). The carrier also desirably contains phosphatidylcholine (PC) or a derivative thereof. Moreover, the carrier does not include targeting agents (such as antibodies).

The carrier typically represents between about 80 wt % of the composition and about 99 wt % of the composition, such as between about 85 wt % and about 95 wt %. Moreover, where the carrier includes both a fatty acid or oil permeation enhancer and PC (and/or PC-derivative), preferably, the weight ratio of permeation enhancer and PC (and/or PC-derivative) is between about 2:1 and 1:2, such as about 1.5:1. The molar ratio of such agents can be, for example, between about 30-35:5-10 (e.g., 33:7), where oleic acid and PC are employed.

The inventive oil-based composition is formulated for topical administration, and, to enhance penetration, most desirably the composition is non-liposomal in nature. Preferably, the composition is an ointment or a cream. Methods for formulating topical oil-based formulations are known to those of skill in the art, and such methods can be employed to formulated the inventive composition. For example, a suitable amount of the PS (and/or PS-derivative) can be admixed with the carrier and blended under suitable conditions to produce the ointment. Alternatively, PS and phosphatidylcholine can be mixed with the carrier (e.g., oleic acid) and mixed completely (as evidenced by a fully transparent product).

In addition to inclusion of PS (and/or PS-derivative) as an active agent, the inventive composition also can contain other (i.e., secondary) active agents. For formulation into the inventive composition, such agent can be dissolved in a suitable solvent (e.g., acetic acid or ethanol or water or chloroform) and added to the composition.

In one embodiment, for example, the composition can contain an aminophospholipid translocase inhibitor (e.g., N-ethyl-maleimide, pyridyldithioethylamine, or combinations thereof). Furthermore, the composition can include a transnitrosylating agent (such as S-nitro-glutathione or S-nitro-cysteine-N-ethyl ether. S-nitro-cysteine-N-ethyl ether is preferred, as it advantageously penetrates cells and effectively causes S-notrosylation of proteins. Without wishing to be bound by theory, it is believed that inclusion of an aminophospholipid translocase inhibitor and/or a transnitrosylating agent can concentrate PS on the outer surface of the tumor cells, enhancing the immune response against such cells. If such agents are desired, N-ethylmaleimide, and S-nitro-glutathione are available commercially (e.g., from Sigma-Aldrich Chemical Co.). S-nitro-cysteine-N-ethyl ether can be prepared as described (Clancy et al., J Med. Chem. 2001, 44(12):2035-8). Also, the effect of pyridyldithioethylamine, a potent inhibitor of the aminophospholipid translocase was described Connor et al., Biochim Biophys Acta. 1991, 1066(1):37-42.

Another type of secondary agent for inclusion in the inventive composition is one or more imidazoquinolines. Members of this class of compounds have been approved for treating several types of skin disorders, including basal cell carcinoma. Any suitable imidazoquinolin can be incorporated into the inventive composition, preferred examples of which include amiquimod (1-[2-methylpropyl]-1H-imidazo[4,5c]quinoline-4-amine), resiquimod (4-amino-α,α-dimethyl-2-ethoxylmethyl-1H-imidazo[4,5-c]-quinoline-1-ethanol), or a combination thereof (See, e.g., Hengge et al., Lancet Infect Dis 2001; 1:189-198; Schiller et al., Exp Dermatol 2006; 15:331-341). Such agents are available from commercial sources, such as 3M Pharmaceutical. Where present, the imidazoquinolin comprises from about 1 wt % to about 10 wt % (e.g., about 5 wt %) of the composition, however, greater or lesser amounts of the imidazoquinolin can be used, as desired.

The composition also can include, as a secondary active agent, an immune response modifier, such as one or more chemokines, cytokines, Toll-like receptor ligands, DC differentiation factors, or a combination thereof. One type of immune response modifier can be a chemokine that serves as a chemoattractant and/or activating factor of DCs. Without wishing to be bound by theory, it is believed that the presence of such chemokine can recruit DCs to the site of the tumor or neoplastic cells, and can also activate the DCs locally at the site of the tumor or neoplastic cells to enhance the immune response against the tumor/neoplasm. Suitable chemokines for inclusion in the inventive composition include those such as an MCP, MIP, and the like (e.g., CCL2, CCL7, CCL8, CCL20, CXCL14, CCL3/MIP-1α, MCP-4/CCL13, VVL5/RANTES, and CCL4/MIP-1β). Such chemokines are available from a wide variety of commercial sources (R&D Systems, Sigma, PeproTech, etc.). Such a chemokine can be included in any suitable concentration, but typically will be present in the composition at a concentration of between about 1 ng/g to 1 mg/g, such as between about 1 μg/g and about 5 μg/g (e.g., about 1 μg/g, 2 μg/g, 4 μg/g, etc.).

Another type of immune response modifier can be a cytokine, for example one that increases the resistance of DCs to tumor-induced inhibition. Without wishing to be bound by theory, it is believed that the inclusion of such cytokines can up-regulate the antitumor potential of the inventive composition by protecting DCs from suppression that is typical of the tumor microenvironment. Suitable cytokines for inclusion in the inventive composition can be one such as are known to those of ordinary skill for achieving this end. For example, such a cytokine can include one or more of IL-2, IL-12, IL-15, IL-18, IL-21, or a combination thereof. Such cytokines are available from a wide variety of commercial vendors (e.g., R&D Systems, Sigma, PeproTech, etc.). Such a cytokine can be included in any suitable concentration, but typically will be present in the composition at a concentration between about 1 ng/g to 1 mg/g such as between 1 μg/g and about 3 μg/g (e.g., about 2 μg/g).

Another type of immune response modifier can be a one or more Toll-like receptor ligands, particularly which activate DC. Desirably, a Toll-like receptor ligand for inclusion in the inventive composition lacks direct effects on tumor cells, but this is not a requirement. Suitable Toll-like receptor ligands for inclusion in the inventive composition include CpG oligodeoxynucleotides and flagellin. Synthetic oligodeoxynucleotides encoding unmethylated CpG motifs (CpG ODN) that trigger an immune reaction are known and can be employed in the context of the present invention (e.g., ODN 2216 (A-class), ODN PF-3512676 (B-class), and ODN 2329 (C-class). Exemplary sequences are: 5′-TCCATGACGTTCCTGATGCT-3′ (SEQ ID NO:1), 5′-TCGATGACGTTCCTGATGCT-3′ (SEQ ID NO:2); 5′-TCGTCGTTTTGTCGTTTTCTCGTT-3′ (SEQ ID NO:3) (See also Uhlmann & Vollmer, J., Curr. Opin. Drug Discov. Devel. 6, 204-217 (2003); Kandimalla et al., Biochem. Soc. Trans. 31, 654-658 (2003)). Moreover, in some embodiments, a Toll-like receptor ligand a TLR-3 ligand (e.g., double-stranded RNA, poly I:C or poly IC:LC) can be employed. Such a Toll-like receptor ligand can be included in any suitable concentration, but typically will be present in the composition at a concentration between about 1 μg/g and about 50 mg/g or between about 1 mg/g and about 30 mg/g. Moreover, the CpG ODN, where included, typically will be included at a higher concentration than flagellin. Thus, the CpG ODN typically will be present at about 20 mg/g (i.e., 40 μg/cm² or 40 μg/application), whereas flagellin typically is included at about 2.5 mg/g (i.e., 5 μg/cm² or 5 μg/application).

Another type of immune response modifier can be a one or more one or more dendritic cell differentiating factors, such as GM-CSF, FLT3L, TNFα, and CD40L. GM-CSF is preferred, as it is a potent inducer and attractant of antigen-presenting cells. Such dendritic cell differentiating factors can be included in any suitable concentration, but typically will be present in the composition at a concentration between about 1 μg/g and about 1 mg/g, such as between about 25 μg/g and about 75 μg/g (e.g., about 50 μg/g). Also, preferably an immune response modifier is not so large as to inhibit penetration through the skin with the carrier.

Without wishing to be bound by theory, it is believed that applying the ointment to the surface of skin permits the PS (and/or PS-derivative) to penetrate the skin and expression of PS on the surface of cancerous cells. In turn, the PS-positive cancerous cells become recognized by antigen-presenting cells (APCs), which can induce antitumor responses leading to the inhibition of tumor/neoplastic growth and/or tumor rejection. Any secondary active agents also can contribute to the antitumor activity by recruiting/protecting/activating DCs or mediating an antitumor effect. Accordingly, the invention provides a method of treating a patient having a neoplastic or cancerous condition by topically applying the inventive composition to the skin of the patient. It will be understood that, while the patient typically is human, the inventive method also can be used in the veterinary context, in which case the patient can be a non-human animal (typically a mammal such as a cat, dog, horse, etc.).

In performing the inventive method, the composition is topically applied to the skin of the patient at a location and in an amount suitable to treat the cancer. The inventive method is particularly suitable to treatment of skin cancers or other neoplastic disorders that manifest in the skin, such as oral cancers, rectal cancers, cancers of the head and neck, epithelial cancers, melanoma, lymphomas (e.g., T-cell lymphomas), etc. For treatment of such cancers/neoplasms, the composition can be applied to an area of the skin overlying the growth or tumor harboring the cancerous/neoplastic cells. The amount of composition, and its frequency of application, can vary depending on the dosage of PS or derivative thereof (as well as any secondary active agent) to be delivered. Typically, the dosage of PS (and/or PS-derivative) is as discussed above.

It should be observed that the inventive method is considered successful even if it does not lead to complete remission of the cancer/neoplasm. In this respect, the inventive method can successfully retard the growth or development of the cancer/neoplasm within the patient. Of course, advantageously, the method results in shrinkage of a tumor or reduction in the number/viability of cancerous or neoplastic cells within the patient. It is most desirable for the inventive method to result in elimination of the tumor. Moreover, the inventive method can be used adjunctively with other treatment regimens (e.g., chemotherapy, radiotherapy, etc).

The following examples further illustrate the invention but, of course, should not be construed as in any way limiting its scope. They demonstrate a clear PS-ointment-induced antitumor effect and rationalize validation of immunomodulating factors, such as cytokines, chemokines, growth factors and BRM, to be included in the PS-ointment composition.

### EXAMPLE 1

This example demonstrates the formulation of an ointment containing PS.

A basic ointment formulation, which consisted of the mixture of a fatty (oleic, C18:1) acid and PC or PC+PS. Oleic acid was utilized as a “solvent” for the phospholipids resulting in the molar ratio of oleic acid:DOPS:DOPC=33:1:7. This is equal to the weight ratio=1.6 g:150 mg:1 g.

DOPS and DOPC were obtained from a commercial source (Avanti Polar Lipids, Alabaster, Ala.). The formulation was prepared by dissolving the DOPS and DOPC in oleic acid.

The basic formulation of the inventive ointment caused significant inhibition of melanoma growth in vivo in B16 mouse tumor models in 3 independent experiments. In vitro studies suggest that the ointment may work through the induction of phagocytosis of PS-expressing LIVE tumor cells by dendritic cells and thus, initiation of antitumor immune responses in mice.

### EXAMPLE 2

This example demonstrates that that topical skin application of the PS-containing ointment is associated with the phospholipid penetration through the skin and incorporation in surrounding cells.

The ointment was prepared in accordance with example 1 except that 20%3 of PS was substituted with fluorescently labeled PS. 1-palmitoyl-2-(6-(7-nitrobenz-2-oxa-1,3-diazol-4-yl)-aminocaproyl) (NBD)-labeled PS (NBD-PS) (Avanti Polar Lipids) was used as the fluorescent probe. B 16 tumor was growing in mice s.c. for 7 days followed by the application of the NBD-PS-ointment twice daily for 3 consecutive days.

After tumor mass incision and triple-enzyme digestion (collagenase, DNase, and hualuronidase), tumor cells were washed and cytospun. For labeling nuclear DNA, cells were preincubated with 1 μg/ml Hoechst (Molecular Probes) at 30° C. for 30 min. Blue and green fluorescent staining was analyzed by confocal microscopy. Green NBD-PS was detected on the surface and inside of many tumor cells. As expected, application of PC- or PC+PS-ointment without NBD yielded no green fluorescence on cells isolated from the tumors. Considering that only 20% of PS in the ointment was labeled with NBD, these data suggest that topical skin application of the PS-containing ointment is associated with the phospholipid penetration through the skin and incorporation in surrounding cells.

### EXAMPLE 3

This example demonstrates that that topical skin application of the PS-containing ointment is associated with the phospholipid penetration through the skin and incorporation in surrounding cells.

The conclusion drawn from Example 2 was confirmed by a direct measurement of phospholipid levels in tumor cells isolated from PS-ointment-treated B16-bearing mice. Topical application of the PS-ointment over the growing tumor was initiated 7 days after s.c. inoculation of B16 cells and 3 days later mice were sacrificed and tumor were harvested. Control group included non-treated animals (3 mice/group). Tumor cells were isolated by a triple enzyme digestion and total lipids were extracted from cells using the Folch procedure [Folch, J Biol Chem 1957; 226:497-509]. The phospholipid classes in the extracts were separated by 2-dimensional HPTLC on silica G plates (5×5 cm, Whatman) as previously described [Tyurina, Antioxid Redox Signal 2004; 6:209-225]. Briefly, the plates were first developed with a solvent system consisting of chloroform:methanol:28% ammonium hydroxide (65:25:5 v/v). After the plates were dried with a forced air to remove the solvent, they were developed in the second dimension with a solvent system consisting of chloroform:acetone:methanol:glacial acetic acid:water (50:20:10:10:5 v/v).

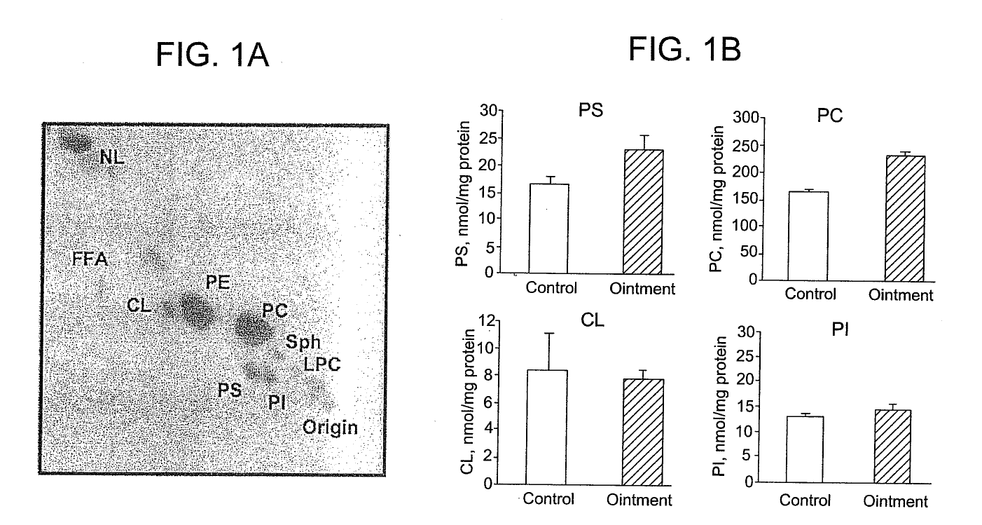

The phospholipids were visualized by exposure to iodine vapor and identified by comparison with migration of authentic phospholipid standards (see FIG. 1A). The spots identified by iodine staining were then scraped and the silicic acid was transferred into the tubes. Lipid phosphorus was determined as described by a micro-method [Bottcher, Anal Chim Acta 1961; 24:203-204]. The identity of each phospholipid was established by comparison with the Rf values measured for the authentic standards. The results of the phospholipid analysis in control and PS-ointment (which contains PC:PS=1:1) treated melanomas from three independent experiments are shown in FIG. 1B. In agreement with the confocal microscopy studies, these data suggest that PS from the topically applied ointment penetrated through the skin and was accumulated in tumor cells.

### EXAMPLE 4

This example demonstrates that topical application of the PS-, but not PC-containing ointment, inhibits tumor growth in vivo.

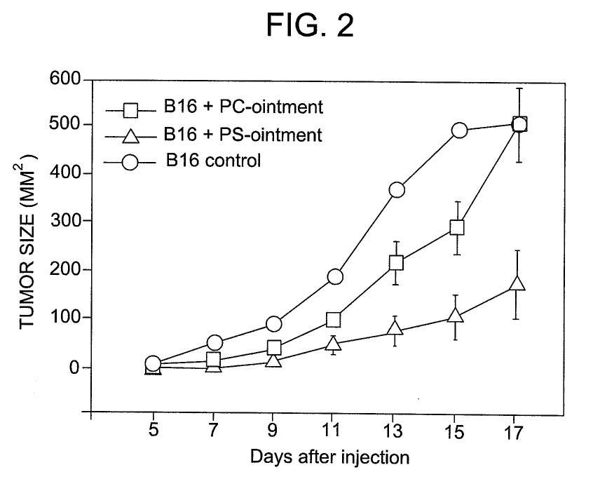

B16 cells (10⁵ cells in 100 μl PBS) were injected s.c. in the right flank in B6 mice and the application of the PC+PS-ointment (termed PS-ointment) was initiated 24 h later. Mice were treated twice/day for 2 weeks. As a control phospholipid ointment, the PC-ointment was used. A negative control included no treatment. This experiment was repeated 3 times with 5-7 animals per group and the mean tumor sizes ±SEM are shown in FIG. 2.

Statistical analysis of the results (Two-way ANOVA for repeated measurements) revealed a significant inhibition of growth of tumors treated with the PS-based, but not the PC-based ointment (p<0.05). These data reveal that local application of the PS-ointment was associated with a strong inhibition of tumor growth.

### EXAMPLE 5

This example demonstrates that PS-ointment induces formation of tumor-specific T cells in B16-bearing mice.

Whether inhibition of tumor growth by the PS-ointment application was associated with the generation of tumor-specific T cells was investigated, since both CD8+ and CD4+ T cells are crucially involved in facilitating antitumor immunity [Ostrand-Rosenberg, Cancer Invest 2005; 23:413-419][Wang, Springer Semin Immunopathol 2005; 27:105-117]. B6 mice (N=6) were injected with B16 cells s.c. (Day 0) and treated with PC- or PC+PS ointments for two weeks (Days 1-14) as described above. Non-treated mice served as a control. Five to seven days later, mice were sacrificed and the spleens were removed. T cells were enriched from the single cell suspensions of splenocytes on the nylon-wool columns and then stimulated with either medium, or irradiated (20,000 rad) B 16 cells or irrelevant RM1 tumor cells (1:5 E:T ratio). Supernatants were collected in 24 h and the levels of IFN-γ and IL-2 were assessed by ELISA.

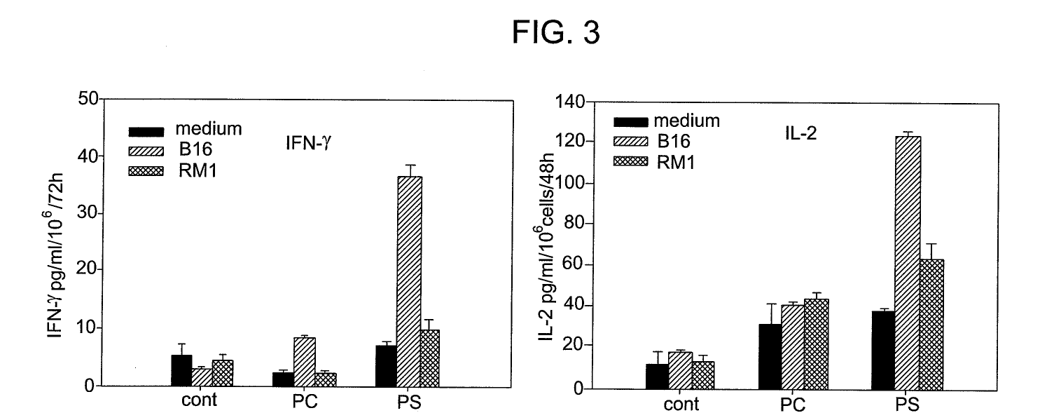

As depicted in FIG. 3, the maximum release of cytokines was observed in T cells obtained from mice treated with the PS-ointment and stimulated with B16 cells (p<0.05 vs control, PC, medium, and RM1 groups; ANOVA). These data demonstrate that application of the PS-ointment in melanoma-bearing mice caused the generation of tumor-specific IFN-γ-producing (presumably CTL) and IL-2-producing (most likely both CD4+ and CD8+) T cells, i.e. suggesting that the treatment is associated with an induction of antitumor immune response.

### EXAMPLE 6

This example demonstrates that PS-ointment-based therapy up-regulates infiltration of the tumor site by leukocytes.

Tumor-infiltrating leukocytes in B16-bearing mice treated with phospholipid-ointments using immunohistochemical (IHC) approach were evaluated as was described earlier [Esche, Cancer Res 1998; 58:380-383]. Both treated and non-treated tumors were removed one week after abrogation of the therapy.

Since the presence of a mononuclear infiltrate might correlate with the intensity of the immune response in a variety of cancers [Whiteside, Semin Cancer Biol 2006; 16:3-15][Witz, Adv Exp Med Biol 2001; 495:317-324], the results of IHC staining of different cell populations, including DC, macrophages, CD4+ and CD8+ cells, were assessed. Tumor-bearing mice were treated with PC- or PS-ointment for 2 weeks and tumors were harvested one week later and frozen in O.C.T. compound. IHC analysis of tumor tissue for infiltration by DC (CD11c), macrophages (F4/80) and CD4+ and CD8+ T cells revealed an increased levels of tumor-associated DC, macrophages and CD8+ T cells in tumors treated with the PS-, but not PC-, ointment. Quantitation of the IHC results by two pathologists revealed that the application of the PC+PS-ointment, but not of the PC-ointment, caused statistically significant increase in tumor-infiltrating DC, macrophages and CD8+ T cells (p<0.05, ANOVA). Although the numbers of CD4+ TIL seem to be higher in the same tissue samples, statistical analysis of these cells was impossible due to the low number of cells in all tested specimens. These IHC data also suggest that the topical PS-ointment therapy is associated with the development of antitumor immune response in tumor-bearing mice.

### EXAMPLE 7

This example demonstrates that PS-ointment-based topical therapy modulates cytokine network in the tumor microenvironment.

A technique which allows determining the levels of cytokines released within the tumor microenvironment in dynamics (up to 10 days) in live freely moving animals was employed. A 10 mm length CMA/20 microdialysis probe (CMA Microdialysis), was implanted and fixed inside of a tumor and extracellular interstitial fluid was collected via the micropump-regulated circulation (16 μl/h) of a buffer (4% (w/v) dextran-70 in PBS). The outer cannula of the probe is made from nanoporous polyethersulfone and has a nominal molecular weight cut-off of 100,000 Da. Multiple samples (˜50 μl) were collected from live animals sitting in specially-designed chambers/cells and the levels of cytokines, chemokines, and growth factors are detected by using the Luminex bead-based multiplexed ELISA. The use of dextran-70 in the circulating buffer helps to counterbalance the high osmolarity of tissue interstitial fluid to prevent probe volume loss and does not interfere with cytokine detection [Rosenbloom, J Immunol Methods 2006; 309:55-68].

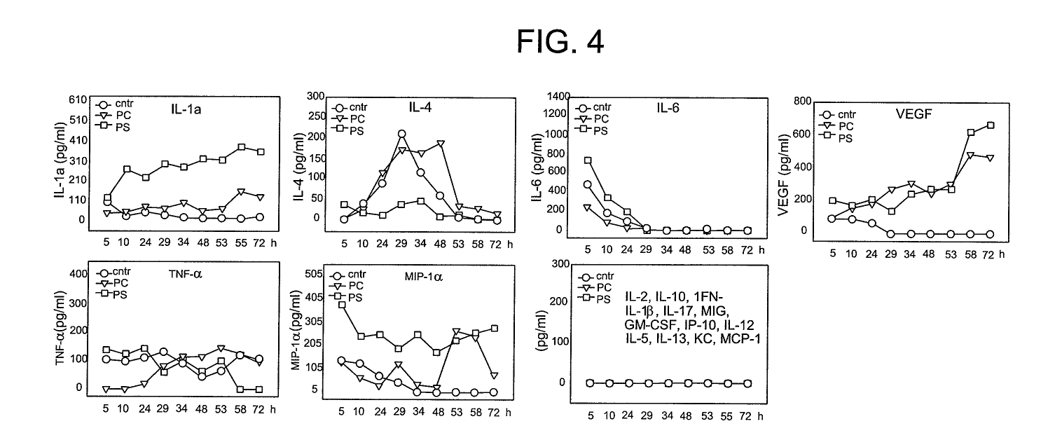

FIG. 4 shows the results of a representative experiment (N=2) when intratumoral cytokines were assessed in specimens harvested for 3 days from the established B 16 tumors during the PC- and PS-ointment-based topical therapy. Non-treated tumor served as an additional control. The application of the PS-ointment was associated with a decrease of intratumoral IL-4 and TNF-α and increase in intratumoral IL-1α and MIP-1α. Unexpectedly, both phospholipid-containing ointments (PC and PC+PS) caused an upregulation of VEGF release, which in the case of PS-ointment, was not associated with an acceleration of tumor growth. These results confirmed the earlier postulation that the cytokine network of many common tumors is rich in inflammatory cytokines, growth factors, and chemokines but generally lacks cytokines involved in specific and sustained immune responses [Burke, Biotherapy 1996; 8:229-241][Negus, World J Urol 1996; 14:157-165]. The data, also demonstrate that the local application of the PS-ointment over the s.c. melanoma may alter the cytokine balance towards more immunogenic and less protumorigenic bias.

### EXAMPLE 8

This example demonstrates that PS-ointment is ineffective in SCID mice and IFN-γ KO mice bearing B 16 melanoma, but induces a systemic effect in immunocompetent mice.

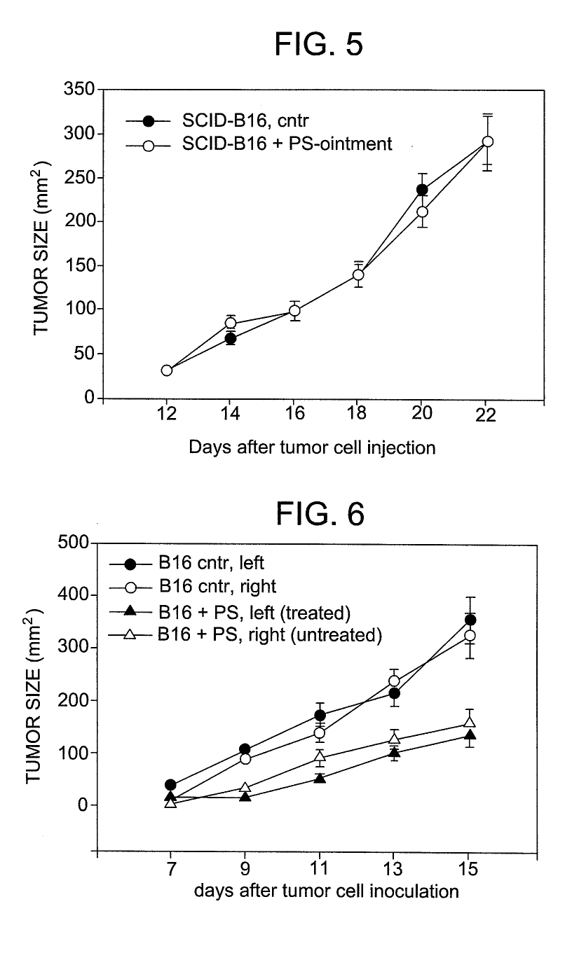

Although the previous Examples support the involvement of the immune system in antitumor activity of the PS-ointment based therapy, additional experiments were conducted to obtain more direct evidence. Whether the PS-ointment inhibited growth of B16 melanoma in lymphocyte-deficient SCID mice was investigated. The design of these studies was similar to that reported in Example 4, except that the group of mice treated with the PC-ointment was omitted and less tumor cells (104/100 μl) were inoculated s.c. FIG. 5 shows that the PS-ointment was unsuccessful in immunodeficient mice, suggesting the role for immune effectors in antitumor activity of the topical PS-ointment based therapy.

Similar studies using IFN-γ knockout mice were conducted. Both control and PS-treated mice survived for less than 10 days after the tumor cell inoculation. The short-term survival of these mice was likely due to the fast spreading of melanoma metastases in the absence of IFN-γ signaling. Thus, these data also confirm the involvement of the immune system in antitumor effect of the PS-ointment based therapy.

To demonstrate the development of systemic antitumor immunity after the topical therapy, additional studies were conducted, in which immunocompetent C57BL6 mice (3 mice/group) received two s.c. B16 cell injections (10⁵ cells in 100 μl PBS) on both right and left flanks, but the PS-ointment was applied over the growing melanoma from Day 2 for 2 weeks only on the left side, i.e. only one tumor was treated. The results revealed that tumor growth was significantly (p<0.05, N=2) inhibited on both sides, although the treated tumor grew slightly slower that their non-treated counterparts (See FIG. 6).

### EXAMPLE 9

This example demonstrates the effect of the addition of immunomodulating components to the basic formulation of the ointment.

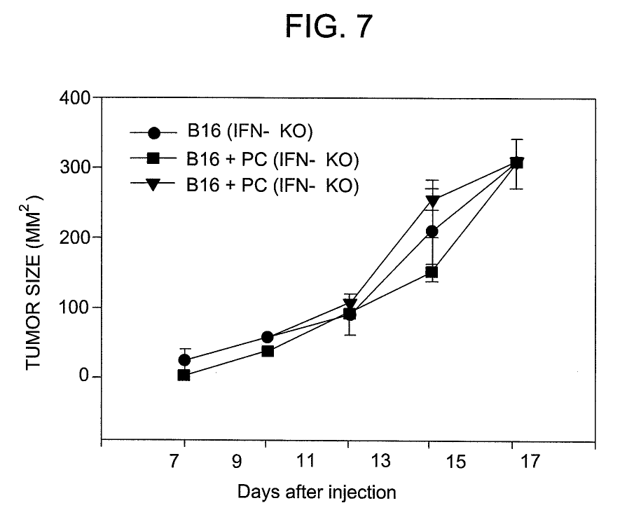

The prior examples demonstrate the involvement of generalized immune response associated with the antitumor activity of the PS-based ointment. This was clearly demonstrated by the lack of antitumor activity of the ointment (no inhibition of tumor growth) in immunodeficient SCID mice. To further elucidate possible mechanisms of antitumor activity of PS-based topical therapy, the effect of interferon gamma (IFN-γ), a key cytokine of antitumor immunity, was assessed for its ability to diminish the antitumor potential of the PS-ointment. To test this experimentally, IFN-γ knockout (KO) mice were inoculated s.c. with B16 melanoma cells and subsequently treated topically with the PS ointment and control PC ointment as described above. Tumor size was determined during the therapy. As shown FIG. 7, either therapy displayed no significant antitumor effect in IFN-γ KO mice.

These results suggest that the effect of the PS-ointment on melanoma growth in mice is mediated, at least in part, by the immune system, i.e. induction of effective and generalized antitumor immune response. Thus, the addition of immunomodulating components to the basic formulation of the ointment is highly justified.

### EXAMPLE 10

This example demonstrates the penetration of PS into the skin of a human patient.

Fresh normal human skin was cut in several 3×3 mm pieces, placed in saline and ointment containing fluorescent NBD-PS was applied onto the skin surface. At different time points thereafter, the skin was harvested, washed, fixed, and analyzed by confocal microscopy for the presence of PS in different layers. Green staining can be seen above the basal membrane after 2 hours of the ointment application and at deeper layers after 3 hours. These results are consistent with the data obtained using mice.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A pharmaceutical composition comprising as an active ingredient phosphatidylserine (PS) or a derivative thereof and a pharmaceutically acceptable oil-based carrier, wherein the composition is formulated for topical administration.

2. The composition of claim 1, which is an ointment or a cream.

3. The composition of claim 1, wherein a derivative of PS comprises two saturated fatty acids, saturated and monounsaturated (oleic, C18:1), or polyunsaturated fatty acids (linoleic C18:2, linolenic C18:3, arachidonic C20:4, docosahexaenoic C20:4) or lyso-forms of such fatty acids.

4. The composition of claim 1, wherein a derivative of PS is an oxidized form of PS or a lyso-form of such oxidized derivative.

5. The composition of claim 1, wherein the PS or a derivative thereof is present in a concentration of from about 1 wt % to about 20 wt % of the composition.

6. The composition of claim 1, wherein the carrier comprises phosphatidylcholine or a derivative thereof.

7. The composition of claim 1, wherein the carrier comprises a fatty acid, a polyunsaturated fatty acid, or a combination thereof.

8. The composition of claim 7, wherein the carrier comprises oleic acid (C18:1), linoleic acid (C18:2), linolenic acid (C18:3), or a combination thereof.

9. The composition of claim 1, comprising an aminophospholipid translocase inhibitor.

10. The composition of claim 9, wherein the aminophospholipid transolcase inhibitor is selected from the group consisting of N-ethyl-maleimide and pyridyldithioethylamine.

11. The composition of claim 1, comprising a transnitrosylating agent.

12. The composition of claim 11, wherein the transnitrosylating agent is S-nitro-glutathione or S-nitro-cysteine-N-ethyl ether.

13. The composition of claim 1, comprising an imidazoquinoline.

14. The composition of claim 13, wherein the imidazoquinolin is amiquimod (1-[2-methylpropyl]-1H-imidazo[4,5c]quinoline-4-amine), resiquimod (4-amino-α,α-dimethyl-2-ethoxylmethyl-1H-imidazo[4,5-c]-quinoline-1-ethanol), or a combination thereof.

15. The composition of claim 13, wherein the imidazoquinolin comprises from about 1 wt % to about 10 wt % of the composition.

16. The composition of claim 1, comprising an immune response modifier.

17. The composition of claim 16, wherein an immune response modifier is selected from the group consisting of chemokines, cytokines, Toll-like receptor ligands, dendritic cell differentiation factors, or a combination thereof.

18. The composition of claim 17, wherein an immune response modifier comprises a chemokine and is present in the composition at a concentration of between about 1 ng/g to 1 mg/g.

19. The composition of claim 17, wherein an immune response modifier comprises a cytokine and is present in the composition at a concentration of between about 1 ng/g to 1 mg/g.

20. The composition of claim 17, wherein the immune response modifier comprises a Toll-like receptor ligands that activate dendritic cells.

21. The composition of claim 20, wherein a Toll-like receptor ligand lacks direct effects on tumor cells.

22. The composition of claim 20, wherein the Toll-like receptor ligand is present at a concentration at between about 1 μg/g and about 50 mg/g.

23. The composition of claim 17, wherein an immune response modifier comprises a dendritic cell differentiating factor.

24. The composition of claim 23, wherein the dendritic cell differentiating factor is present in the composition at a concentration of between about 1 μg/g and about 1 mg/g.

25. A method of treating a neoplastic disorder in a human patient comprising topically applying to the human patient a pharmaceutical composition in an amount and at a location sufficient to treat the neoplastic disorder within the human patient, the composition comprising as an active ingredient phosphatidylserine (PS) or a derivative thereof and a pharmaceutically acceptable oil-based carrier and wherein the neoplastic disorder is oral cancer, rectal cancer, head and neck cancer, epithelial cancer, melanoma, basal cell carcinoma, squamous cell carcinoma, or lymphoma.

26. The method of claim 25, wherein the composition is an ointment or a cream.

27. The method of claim 25, wherein the derivative of PS comprises two saturated fatty acids, saturated and monounsaturated (oleic, C18:1), or polyunsaturated fatty acids (linoleic C18:2, linolenic C18:3, arachidonic C20:4, docosahexaenoic C20:4) or lyso-forms of such fatty acids.

28. The method of claim 25, wherein the derivative of PS is an oxidized form of PS or a lyso-form of such oxidized derivative.

29. The method of claim 25, wherein the PS or a derivative thereof is present in a concentration of from about 1 wt % to about 20 wt % of the composition.

30. The method of claim 25, wherein the composition is applied to deliver PS or a derivative thereof in a dosage between about 10 μg/cm²/day and about 75 μg/cm²/day.

31. The method of claim 25, wherein the carrier comprises phosphatidylcholine or a derivative thereof.

32. The method of claim 25, wherein the carrier comprises a fatty acid, a polyunsaturated fatty acid, or a combination thereof.

33. The method of claim 32, wherein the carrier comprises oleic acid (C18:1), linoleic acid (C18:2), linolenic acid (C18:3), or a combination thereof.

34. The method of claim 25, wherein the composition comprises an aminophospholipid translocase inhibitor.

35. The method of claim 34, wherein the aminophospholipid transolcase inhibitor is selected from the group consisting of N-ethyl-maleimide and pyridyldithioethylamine.

36. The method of claim 25, wherein the composition comprises a transnitrosylating agent.

37. The method of claim 36, wherein the transnitrosylating agent is S-nitro-glutathione or S-nitro-cysteine-N-ethyl ether.

38. The method of claim 25, wherein the composition comprises an imidazoquinoline.

39. The method of claim 28, wherein the imidazoquinolin is amiquimod (1-[2-methylpropyl]-1H-imidazo[4,5c]quinoline-4-amine), resiquimod (4-amino-α,α-dimethyl-2-ethoxylmethyl-1H-imidazo[4,5-c]-quinoline-1-ethanol), or a combination thereof.

40. The method of claim 38, wherein the imidazoquinolin comprises from about 1 wt % to about 10 wt % of the composition.

41. The method of claim 25, wherein the composition comprises an immune response modifier.

42. The method of claim 41, wherein an immune response modifier is selected from the group consisting of chemokines, cytokines, Toll-like receptor ligands, dendritic cell differentiation factors, or a combination thereof.

43. The method of claim 42, wherein an immune response modifier comprises a chemokine and is present in the composition at a concentration of between about 1 ng/g to 11 mg/g.

44. The method of claim 42, wherein an immune response modifier comprises a cytokine and is present in the composition at a concentration of between about 1 ng/g to 1, mg/g.

45. The method of claim 42, wherein the immune response modifier comprises a Toll-like receptor ligand that activates dendritic cells.

46. The method of claim 45, wherein a Toll-like receptor ligand lacks direct effects on tumor cells.

47. The method of claim 45, wherein the Toll-like receptor ligand is present at a concentration at between about 1 μg/g and about 50 mg/g.

48. The method of claim 42, wherein an immune response modifier comprises a dendritic cell differentiating factor.

49. The method of claim 48, wherein the dendritic cell differentiating factor is present in the composition at a concentration of between about 1 μg/g and about 1 mg/g.


SEQUENCE LISTING (3 sequences)

SEQ ID NO: 1 - DNA, 20 bp, Artificial
  oligodeoxynucleotide encoding unmethylated CpG motif
tccatgacgttcctgatgct

SEQ ID NO: 2 - DNA, 20 bp, Artificial
  oligodeoxynucleotide encoding unmethylated CpG motif
tcgatgacgttcctgatgct

SEQ ID NO: 3 - DNA, 24 bp, Artificial
  oligodeoxynucleotide encoding unmethylated CpG motif
tcgtcgttttgtcgttttctcgtt